(19) United States
(12) Patent Application Publication
(10) Pub. No.: US 2023/0277237 A1
(43) Pub. Date: Sep. 7, 2023

(54) KNIFE HEAD ASSEMBLY OF A NOVEL MEDICAL ELECTROTOME AND A NOVEL MEDICAL ELECTROTOME

(71) Applicant: LEO MEDICAL CO.,LTD., Changzhou City (CN)

(72) Inventors: Peng KOU, Changzhou City (CN); Quanbo GE, Changzhou City (CN); Zhongmin ZHANG, Changzhou City (CN)

(21) Appl. No.: 17/686,554

(22) Filed: Mar. 4, 2022

(51) Int. Cl. A61B 18/14; A61B 18/12

(52) U.S. Cl. CPC A61B 18/1492; A61B 18/1206; A61B 2018/1407; A61B 2018/122; A61B 2018/00982

(57) ABSTRACT

The present invention provides a knife head assembly of a novel medical electrotome and a novel medical electrotome. The knife head assembly comprises a snare ring and a cutting knife, the cutting knife comprises a knife handle fixedly connected to a distal end of the snare ring and a knife head fixedly connected to a distal end of the knife handle; the surface of the knife handle is provided with two grooves that are arranged symmetrically and extend in a length direction of the knife handle, the knife handle is further provided with a through-hole penetrating through the knife handle, the two ends of the through-hole are in communication with the two grooves respectively, the distal end of the snare ring is inserted through the through-hole, and the parts of the snare ring on the two sides of the through-hole are limited in the grooves, thus the distal end of the snare ring is embedded and fixed in the knife handle. In the present invention, the snare ring and the cutting knife are mechanically fixed in an embedded manner, and the distal end of the snare ring is inserted and embedded in the knife handle of the cutting knife; the fixed connection structure is stable and reliable, effectively prevents the cutting knife from falling off from the distal end of the snare ring accidentally, and thereby improves the safety of surgical operations.

### TECHNICAL FIELD

The present invention relates to the field of medical equipment, in particular to a knife head assembly of a novel medical electrotome and a novel medical electrotome, the novel medical electrotome is used for excision or dissection of polyps or tumor mucosae in the digestive tract or respiratory tract.

### BACKGROUND ART

Endoscopic submucosal dissection (ESD) is a new treatment means emerged in recent years as well as a technique with a great clinical application prospect, which enables one-off complete resection of more early-stage digestive tract carcinoids under an endoscope and avoids the pain of laparotomy and resection of organs. Compared with laparotomy and previous endoscopic treatment method such as endoscopic mucosal resection (EMR), ESD has advantages, for example, the wound is smaller, the patient can receive multiple treatments at multiple sites, the doctor can obtain complete histopathological specimens for analysis, and the resection ratio of tumors having large area and irregular shape or tumors accompanied with ulcers and scars can reach up to 96% or higher, so as to decrease the probability of recurrence.

A conventional ESD surgical process involves operations such as marking resection range, injection bulge, mucosal incision and dissection, and hemostasis, etc., and involves a variety of instruments, such as Dull Knife, IT Knife, Hook Knife, syringe needles, hemostatic clips, hot biopsy forceps, etc., with a high total cost; in addition, various instruments have to be replaced frequently in the surgical process, and the surgical process is time-consuming.

Presently, the Chinese Invention Patent Application No. 201510506373.0 has disclosed a medical electroresection and electrocoagulation device, and the Chinese Invention Patent Application No. 201610423834.2 has disclosed a rotary electroresection and electrocoagulation device. Though the two medical instruments can partially replace the above-mentioned instruments used in the ESD surgical process for marking resection range, mucosal incision and dissection, and hemostasis, but are inapplicable for injection bulge. Therefore, instruments such as syringe needles and the like still have to be exchanged in the ESD surgical process for injection bulge. Besides, in the above two existing techniques, the clearance between the traction rope and the outer sheath tube is large, consequently the head end of the snare ring may project out and become unstable, and misoperation may occur when peripheral electroresection and electrocoagulation is performed.

Furthermore, on some medical electrotomes in the prior art, a cutting knife is arranged at the distal end of the snare ring, and the cutting knife and the snare ring are fixed together by crimping or welding. However, such a fixed connection mode is not very reliable, and a severe adverse consequence may be resulted once the cutting knife falls off accidentally during the operation.

### CONTENTS OF THE INVENTION

In view of the above-mentioned drawbacks in the prior art, the object of the present invention is to provide a knife head assembly of a novel medical electrotome, which realizes a reliable fixed connection between the cutting knife and the snare ring, and effectively prevents the cutting knife from falling off accidentally.

To attain the above object, the present invention provides a knife head assembly of a novel medical electrotome. The knife head assembly comprises a snare ring and a cutting knife, the cutting knife comprises a knife handle fixedly connected to a distal end of the snare ring and a knife head fixedly connected to a distal end of the knife handle; the surface of the knife handle is provided with two grooves that are arranged symmetrically and extend in a length direction of the knife handle, the knife handle further is provided with a through-hole penetrating through the knife handle, the two ends of the through-hole are in communication with the two grooves respectively, the distal end of the snare ring is inserted through the through-hole, and the parts of the snare ring on the two sides of the through-hole are limited in the grooves, thus the distal end of the snare ring is embedded and fixed in the knife handle.

Furthermore, the distal end of the snare ring is integrally provided with a U-shaped ring connection portion, the ring connection portion comprises a first bent section, a second bent section and a third bent section that are connected sequentially, the second bent section is inserted through the through-hole, and the first bent section and the third bent section are received in the two grooves respectively.

Furthermore, the snare ring is formed by multiple strands of braided wires made of stainless steel, or multiple strands of braided wires made of nickel and titanium, or multiple strands of braided wires made of a composite material of stainless steel, nickel and titanium, or a single wire made of stainless steel, or a single wire made of nickel and titanium.

Furthermore, the snare ring and the knife handle are further fixed together by welding or bonding.

Furthermore, the cutting knife is an integrally formed part.

Furthermore, the knife head is of a mushroom-head type, a triangular type, a doornail type, an L-type or a linear type.

The present application further provides a novel medical electrotome, which comprises an outer sheath tube, a traction rope movably inserted in the outer sheath tube, a handle assembly for driving the traction rope to move, and the knife head assembly described above, wherein a proximal end of the snare ring is fixedly connected with a distal end of the traction rope.

Furthermore, the handle assembly comprises a handle holder fixed with a proximal end of the outer sheath tube, and an operating handle in sliding fit with the handle holder in the extension direction of the outer sheath tube; the novel medical electrotome further comprises a catheter movably inserted in the handle holder and fixed with a proximal end of the traction rope, a protective tube fixed to a distal end of the outer sheath tube, and an electrocoagulation electrode fixed inside the outer sheath tube; the handle holder is provided with a gas-liquid connector thereon and a gas-liquid channel therein, wherein the gas-liquid connector is configured to be connected to a gas source or liquid source, and the gas-liquid channel, an inner cavity of the outer sheath tube, and an inner cavity of the protective tube are in communication sequentially; the operating handle is provided with an energized connector connected with the catheter, wherein the energized connector is configured to connect to a power source, and all of the catheter, the traction rope and the snare ring are electrically conductive; the electrocoagulation electrode is arranged at a distal section of the outer sheath tube and adjacent to a proximal end of the protective tube, and the snare ring can contact and cooperate with the electrocoagulation electrode when the snare ring is drawn into the outer sheath tube.

Furthermore, the novel medical electrotome further comprises a connecting tube arranged inside the outer sheath tube, wherein the connecting tube is disposed between the snare ring and the traction rope, and a distal end and a proximal end of the connecting tube are fixedly connected with the proximal end of the snare ring and the distal end of the traction rope respectively.

Furthermore, the novel medical electrotome further comprises a star-shaped tube arranged inside the outer sheath tube and sleeved on the traction rope, and limiting tubes that abut against the two ends of the star-shaped tube and are fixed to the traction rope, wherein ribs are arranged on the outer circumferential surface of the star-shaped tube, and the maximum outer diameter of the star-shaped tube is smaller than the inner diameter of the outer sheath tube.

Furthermore, the protective tube comprises a hollow cylindrical section extending into the outer sheath tube, and a hollow conical section integrally extending out of a distal end of the hollow cylindrical section and located outside the outer sheath tube, wherein the protective tube has a maximum outer diameter at a proximal end of hollow conical section and the maximum outer diameter is equal to the outer diameter of the outer sheath tube, and the outer surface of the hollow cylindrical section is provided with protrusions tightly fitting with the inner wall of the outer sheath tube.

Furthermore, the handle holder has an energized channel that is arranged at the energized connector and receives part of the catheter, and has a sealing device fixed therein for isolating the energized channel from the gas-liquid channel, wherein the catheter is movably inserted in the sealing device.

Furthermore, the handle assembly further comprises a locking assembly; the operating handle has a through-hole perpendicular to a sliding direction of the operating handle; the handle holder has an open slot extending in the sliding direction of the operating handle; the locking assembly comprises a rotating rod inserted in the through-hole, and end of the rotating rod is located in the open slot and has two opposite long planar sides, and the length of the long sides is greater than the length of the other sides; when the other sides of the rotating rod are oriented in the width direction of the opening of the open slot, the operating handle can move axially in relation to the handle holder; when the rotating rod is rotated so that the long sides of the rotating rod are oriented in the width direction of the opening of the open slot, the rotating rod will enlarge the opening degree of the open slot, so that the outer wall surface of the handle holder firmly abut against the wall surface of the operating handle with through-hole, thereby the handle holder and the operating handle are tightly locked.

As described above, the knife head assembly of the novel medical electrotome and the novel medical electrotome in the present invention attain the following beneficial effects:

In the present invention, the snare ring and the cutting knife are mechanically fixed in an embedded manner, and the distal end of the snare ring is inserted and embedded in the knife handle of the cutting knife; the fixed connection structure is stable and reliable, effectively prevents the cutting knife from falling off from the distal end of the snare ring accidentally, and thereby improves the safety of surgical operations.

### BRIEF DESCRIPTION OF DRAWINGS

FIG. 1 is a schematic structural diagram of the novel medical electrotome in the present application;

FIG. 2 shows the state diagram of the distal end of the novel medical electrotome in the present application when the novel medical electrotome performs electrocoagulation operation;

FIG. 3 shows the state diagram of the distal end of the novel medical electrotome in the present application when the novel medical electrotome performs margin electroresection and electrocoagulation operation;

FIG. 4 is a schematic structural diagram of the electrocoagulation electrode the present application;

FIGS. 5a-5d are schematic structural diagrams of four examples of the protective tube in the present application and the schematic structural diagrams are sectional views;

FIGS. 6a-6b are schematic structural diagrams of two examples of the star-shaped tube in the present application and the schematic structural diagrams are side views;

FIG. 7 is a local schematic structural diagram of the novel medical electrotome in the present application at the handle holder;

FIG. 8 is a schematic structural diagram of the knife head assembly in the present application;

FIGS. 9-11 are schematic structural diagrams of the cutting knife in the present application from different viewing angles;

FIGS. 12a-12f are schematic structural diagrams of four examples of the knife head of the cutting knife in the present application, wherein FIG. 12f is a distal side view of FIG. 12e;

FIG. 13 is a schematic structural diagram of the handle assembly in the present application;

FIG. 14 is a schematic structural diagram of the rotating rod in the present application;

FIG. 15 is a schematic structural diagram of the rotating block in the present application;

FIG. 16 is a local schematic structural diagram of the operating handle in the present application;

FIG. 17 is a schematic diagram of the fitting between the rotating rod and the open slot in the handle holder in the present application;

FIG. 18 shows a state diagram in which the rotating rod and the operating handle can move in relation to each other in the present application;

FIG. 19 is a sectional view of FIG. 18;

FIG. 20 shows a state diagram in which the rotating rod is tightly locked in relation to the operating handle in the present application; and

FIG. 21 is a sectional view of FIG. 20.

### DESCRIPTION TO THE REFERENCE NUMBERS OF ELEMENTS

1—snare ring, 101—ring connection portion, 102—first bent section, 103—second bent section, 104—third bent section, 2—connecting tube, 3—protective tube, 301—hollow cylindrical section, 302—hollow conical section, 303—protrusion, 4—outer sheath tube, 5—electrocoagulation electrode, 501—helical spring tube, 502—pin, 6—traction rope, 7—handle holder, 701—gas-liquid channel, 702—energized channel, 8—sealing device, 9—catheter, 10—gas-liquid connector, 11—energized connector, 12—operating handle, 13—high frequency power source, 14—gas storage tank, 15—syringe, 16—cutting knife, 161—knife handle, 162—knife head, 163—groove, 164—knife connecting portion, 165—through-hole, 17—limiting tube, 18—star-shaped tube, 181—rib, 19—pressing block, 20—rotating rod, 21—rotating block.

### DETAILED EMBODIMENTS

Hereunder, the present invention will be described in specific examples below, and those skilled in the art can easily understand other advantages and effects of the present invention from the disclosure in this specification.

It should be noted that the structures, proportions, sizes, etc. depicted in the accompanying drawings of this specification are only intended to be in coordination with the disclosure of this specification to facilitate those skilled in the art to understand and read, but are not intended to define the restrictive conditions for implementation of the present invention, and thus do not have any substantive technical meaning. Any modification to the structures, change to the proportional relations or adjustment to the sizes, made without affecting the effects or objects that can be attained by the present invention, shall still be deemed as falling in the scope covered by the technical content disclosed in the present invention. Besides, the terms such as “top”, “bottom”, “left”, “right”, “middle”, “a” or “an” and the like quoted in this specification are used only to facilitate clear description rather than define the scope of implementation of the present invention. Any change or adjustment to the relative relations of the terms without substantially changing the technical content shall be deemed as falling in the scope of implementation of the present invention.

The present application provides a medical instrument, particularly a novel medical electrotome for excision or dissection of polyps or tumor mucosa in the digestive tract or respiratory tract, and a knife head assembly of the novel medical electrotome. In order to describe the structures of the present application more clearly, orientation terms such as “distal end” and “proximal end” are used in the following examples, wherein the term “distal end” refers to an end of the instrument away from the operator in the surgical process, while the term “proximal end” refers to an end of the instrument near the operator in the surgical process.

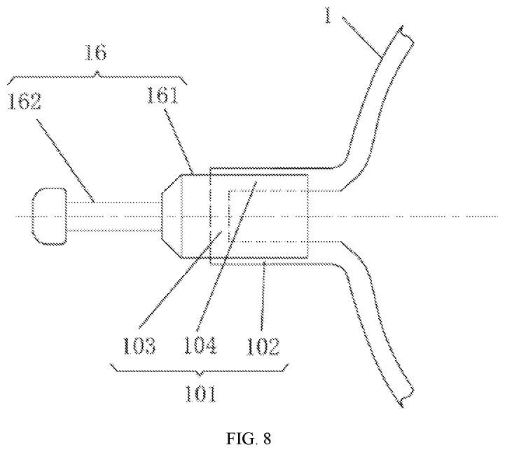

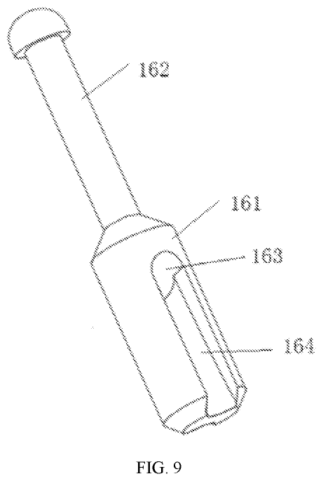

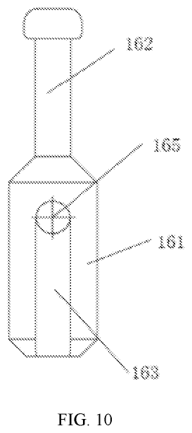

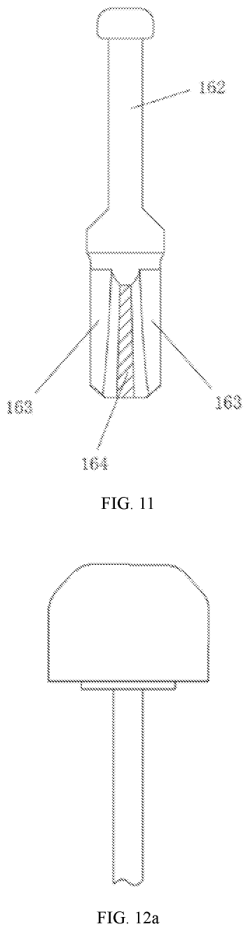

As shown in FIG. 8, the knife head assembly of the novel medical electrotome in the present application comprises a snare ring 1 and a cutting knife 16; as shown in FIGS. 9-11, the cutting knife 16 comprises a knife handle 161 fixedly connected with a distal end of the snare ring 1, and a knife head 162 fixedly connected with a distal end of the knife handle 161, wherein the surface of the knife handle 161 is provided with two grooves 163 symmetrically arranged and extending in a length direction of the knife handle 161, and the knife handle 161 is further provided with a through-hole 165 penetrating through the knife handle 161, and the two ends of the through-hole 165 are in communication with the two grooves 163 respectively; the distal end of the snare ring 1 is inserted through the through-hole 165, and the parts of the snare ring 1 on the two sides of the through-hole 165 are partially limited in the grooves 163, thus the distal end of the snare ring 1 is embedded and fixed in the knife handle 161.

As shown in FIG. 1, the novel medical electrotome in the present application comprises a hollow outer sheath tube 4, a traction rope 6 movably inserted in the outer sheath tube 4, a handle assembly for driving the traction rope 6 to move, and the above-mentioned knife head assembly, wherein a proximal end of the snare ring 1 in the knife head assembly is fixedly connected with a distal end of the traction rope 6. When the handle assembly drives the traction rope 6 to move back and forth in the outer sheath tube 4, the traction rope 6 drives the knife head assembly to move back and forth together, thereby the snare ring 1 and the cutting knife 16 extend out of the outer sheath tube 4 or are drawn into the outer sheath tube 4.

In the present invention, the snare ring 1 and the cutting knife 16 are mechanically fixed in an embedded manner, and the distal end of the snare ring 1 is inserted and embedded in the knife handle 161 of the cutting knife 16; the fixed connection structure is stable and reliable, effectively prevents the cutting knife 16 from falling off from the distal end of the snare ring 1 accidentally, and thereby improves the safety of surgical operations.

Furthermore, as shown in FIGS. 9-11, the knife handle 161 of the cutting knife 16 has a knife connecting portion 164 located between the two grooves 163, and a through-hole 165 is arranged in the distal end of the knife connecting portion 164, i.e., arranged in the distal end of the groove 163; as shown in FIG. 8, the distal end of the snare ring 1 is integrally provided with a U-shaped ring connection portion 101, the ring connection portion 101 comprises a first bent section 102, a second bent section 103 and a third bent section 104 that are connected sequentially, the second bent section 103 is inserted through the through-hole 165, and the first bent section 102 and the third bent section 104 are received in the two grooves 163 respectively. Thus, the snare ring 1 is embedded in the knife connecting portion 164 of the knife handle 161 via the U-shaped ring connection portion 101. In the process of connecting the cutting knife 16 with the snare ring 1, the snare ring 1 is inserted into the through-hole 165 of the cutting knife 16 first, then the distal end of the snare ring 1 is bent into a first bent section 102, a second bent section 103 and a third bent section 104 sequentially, so that the snare ring 1 can be embedded and fixed in the handle 161 of the cutting knife 16, and the distal end of the snare ring 1 is formed in a tip configuration at the same time. Preferably, the snare ring 1 and the knife handle 161 are further fixed by welding or bonding, so as to further ensure the reliability of the fixed connection between the snare ring 1 and the knife handle 161.

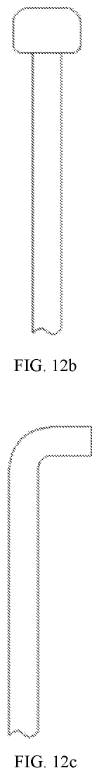

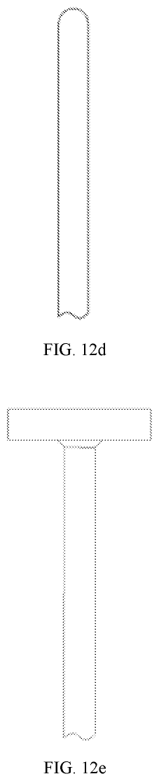

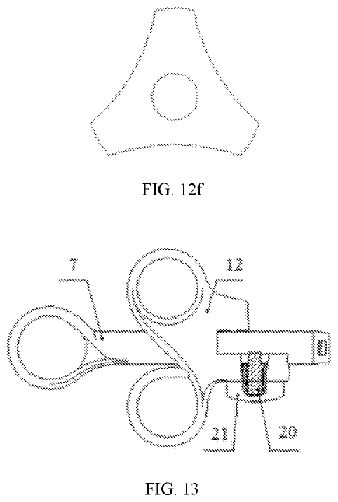

Furthermore, the cutting knife 16 is an integrally formed part. The knife head 162 is a cutting portion of the cutting knife 16, and may be in a variety of shapes, such as mushroom-head shape as shown in FIG. 12a, a doornail shape as shown in FIG. 12b, an L shape as shown in FIG. 12c, a linear shape as shown in FIG. 12d, or a triangular shape as shown in FIGS. 12e and 12f. The cutting knife 16 having a suitable knife head 162 may be selected according to the requirement for effective resection of different pathological tissues in the surgical process and the operating habit of the doctor.

Furthermore, the snare ring 1 may be formed by multiple strands of braided wires made of stainless steel, or multiple strands of braided wires made of nickel and titanium, or multiple strands of braided wires made of a composite material of stainless steel, nickel and titanium, or a single wire made of stainless steel, or a single wire made of nickel and titanium, to ensure that the head at the distal end will not deform or be damaged after long-time energized resection; in addition, the snare ring 1 has certain rigidity and can be kept stable when it is used as an acupotome.

Furthermore, a preferred example of the novel medical electrotome is as follows: as shown in FIG. 1, the handle assembly comprises a handle holder 7 fixed with the proximal end of the outer sheath tube 4, and an operating handle 12 in sliding fit with the handle holder 7 in the extension direction of the outer sheath tube 4, and the operating handle 12 can slide in relation to the handle holder 7 in the extension direction of the outer sheath tube 4; in addition to the above-mentioned snare ring 1, cutting knife 16, outer sheath tube 5, traction rope 6, handle holder 7 and operating handle 12, the novel medical electrotome further comprises a catheter 9, a protective tube 3 and an electrocoagulation electrode 5. Specifically, the catheter 9 is movably inserted in the handle holder 7, and the catheter 9 is fixedly connected with the proximal end of the traction rope 6, the protective tube 3 is fixedly connected with the distal end of the outer sheath tube 4, and the electrocoagulation electrode is arranged at the distal end section of the outer sheath tube 4 and fixed inside the outer sheath tube 4 and is adjacent to the proximal end of the protective tube 3. The handle holder 7 is provided with a gas-liquid connector 10 thereon and a gas-liquid channel 701 therein, wherein the gas-liquid connector 10 is configured to be connected with a gas source (e.g., a gas storage tank 14) or a liquid source (e.g., a syringe 15), and the gas-liquid channel 701, an inner cavity of the outer sheath tube 4 and an inner cavity of the protection tube 3 are in communication sequentially in the direction from the distal end to the proximal end. The operating handle 12 is provided with an energized connector 11 connected with the catheter 9, the energized connector 11 is connected to a power source (e.g., a high frequency power source 13); all of the catheter 9, the traction rope 6 and the snare ring 1 are electrically conductive.

With the novel medical electrotome described above, when the operator moves the operating handle 12 at the distal end, the operating handle 12 drives the traction rope 6 to move via the catheter 9, the traction rope 6 drives the snare ring 1 and the cutting knife 16 to move together, thus the cutting knife 16 and the snare ring 1 are pushed out of the protective tube 3, and the cutting knife 16 and the snare ring 1 are drawn into the outer sheath tube 4. When the operating handle 12 is pushed forward, the traction rope 6 drives the snare ring 1 and the cutting knife 16 to move together in a direction toward the distal end; when the operating handle 12 is pulled backward, the traction rope 6 drives the snare ring 1 and the cutting knife 16 together to move in a direction toward the proximal end. Preferably, as shown in FIG. 1, the novel medical electrotome further comprises a connecting tube 2 arranged inside the outer sheath tube 4 and disposed between the snare ring 1 and the traction rope 6, wherein a distal end and a proximal end of the connecting tube 2 are fixedly connected with the proximal end of the snare ring 1 and the distal end of the traction rope 6 respectively, for example, by compression joint, welding, or bonding, etc. The novel medical electrotome described above has injection function, electroresection function, electrocoagulation function, and margin electroresection and electrocoagulation function.

The gas-liquid connector 10, the handle holder 7, the catheter 9, the gas-liquid channel 701, the outer sheath tube 4 and the protective tube 3 constitute an injection unit of the novel medical electrotome. When the injection function of the novel medical electrotome is enabled, the operating handle 12 may be pulled backward to draw the cutting knife 16 and the snare ring 1 into the outer sheath tube 4; the distal end of the protective tube 3 abuts against a small incision preformed in the mucosal tissue, the syringe 15 is connected at the gas-liquid connector 10 for liquid injection, the liquid flows out through the gas-liquid channel 701, the inner cavity of the outer sheath tube 4 and the protective tube 3, so that the injection bulge operation is performed. Alternatively, if the viewing field is blocked by blood or other filth in the surgical process, physiological saline may be injected with the syringe 15 through the gas-liquid connector 10 on the handle holder 7, and then the physiological saline is sprayed out from a distal orifice of the protective tube 3 to rinse out the area in the viewing field, thus a rinsing operation in the surgical process is performed.

The energized connector 11, the operating handle 12, the catheter 9, the handle holder 7, the outer sheath tube 4, the traction rope 6, the connecting tube 2, the snare ring 1 and the cutting knife 16 constitute an electroresection unit of the novel medical electrotome. When the electroresection function of the novel medical electrotome is enabled, the operating handle 12 may be pushed forward so that the knife head 162 at the distal end of the cutting knife 16 extends out of the protective tube 3 completely, the energized connector 11 is connected to the high frequency power source 13, and current flows through the energized connector 11, the catheter 9, the traction rope 6 and the snare ring 1 sequentially to the cutting knife 16, thus an electroresection operation may be performed. For example, snare resection operation of polyps or tissues or snare removal operation of foreign matters may be carried out.

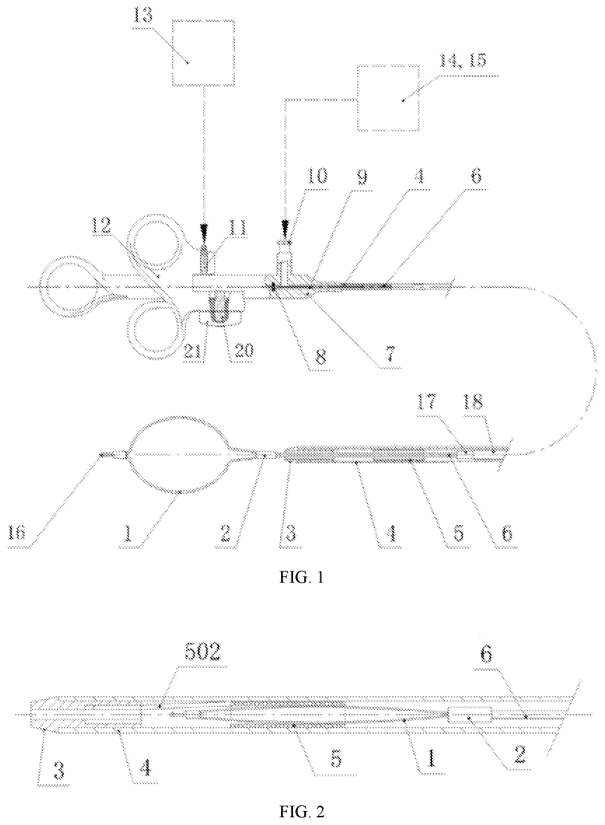

The energized connector 11, the operating handle 12, the catheter 9, the gas-liquid connector 10, the handle holder 7, the gas-liquid channel 701, the outer sheath tube 4, the traction rope 6, the connecting tube 2, the snare ring 1, the cutting knife 16, the protective tube 3, and the electrocoagulation electrode 5 constitute an electrocoagulation unit of the novel medical electrotome. When the electrocoagulation function of the novel medical electrotome is enabled, the operating handle 12 may be pulled backward, so that the cutting knife 16 and the snare ring 1 are drawn into the outer sheath tube 4 completely; at that point, as shown in FIG. 2, the snare ring 1 contacts and cooperates with the electrocoagulation electrode 5; the energized connector 11 is connected to the high frequency power source 13, the gas-liquid connector 10 is connected to the gas storage tank 14 filled with inert gas, and the gas storage tank 14 supplies the inert gas, the inert gas is conveyed through the gas-liquid channel 701 and the inner cavity of the outer sheath tube 4 sequentially to the electrocoagulation electrode 5, and the insert gas is ionized at the electrocoagulation electrode 5; the ionized gas is sprayed through the protective tube 3 to the surface of the tissue at a high speed to achieve blood coagulation for hemostasis; thus an electrocoagulation operation is performed. Here, the electrocoagulation refers to blood coagulation for hemostasis by means of ionization of inert gas. For example, by means of ionization of argon gas at the electrocoagulation electrode 5, marking of the operation range on the tissue or blood coagulation on the wound surface for hemostasis is carried out.

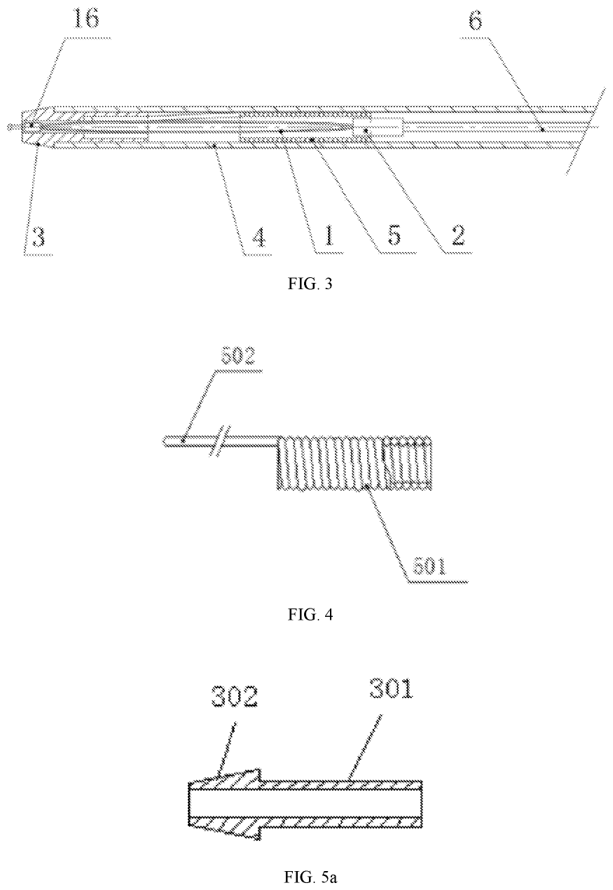

The energized connector 11, the operating handle 12, the catheter 9, the handle holder 7, the outer sheath tube 4, the traction rope 6, the connecting tube 2, the snare ring 1 and the cutting knife 16 constitute a margin electroresection and electrocoagulation unit of the novel medical electrotome. When the margin electroresection and electrocoagulation function of the novel medical electrotome is enabled, the operating handle 12 may be pushed forward, so that the cutting knife 16 and the cutting portion at the distal end of the snare ring 1 extend out from the protective tube 3, as shown in FIG. 3, the energized connector 11 is connected to the high frequency power source 13, and the cutting portion at the distal end of the snare ring 1 carries out the margin resection and electrocoagulation operation. Here, the electrocoagulation refers to that the metallic cutting portion of the snare ring 1 is energized and cauterizes the tissue at a high temperature for blood coagulation and hemostasis. For example, when margin resection and electrocoagulation is carried out, mucosal tissue incision, local electrocoagulation and cauterization can be performed for hemostasis.

Therefore, the novel medical electrotome involved in the present application can perform injection operation, electroresection operation, electrocoagulation operation, and margin electroresection and electrocoagulation operation; thus, the operator can accomplish an ESD surgical operation completely, independently and safely, avoid exchange of instruments such as syringe needles used in injection bulge, thereby reduces the surgery cost and operation time of ESD operation.

Preferably, as shown in FIG. 4, the electrocoagulation electrode 5 comprises a section of helical spring tube 501, and a pin 502 extending from the distal end of the helical spring tube 501 in a direction toward the protective tube 3 and to an accessory of the protective tube 3. As shown in FIG. 2, the electrocoagulation electrode 5 is spaced from the protective tube 3 by a distance within a range of 0 to 50 mm, and the electrocoagulation electrode 5 is closer to the operating handle 12 than the protective tube 3.

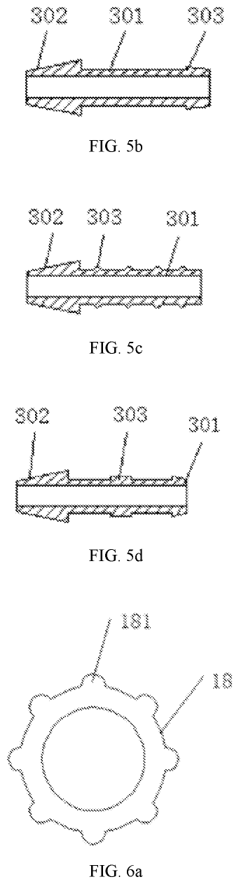

Furthermore, as shown in FIGS. 2 and 5a, the protective tube 3 comprises a hollow cylindrical section 301 extending into the outer sheath tube 4, and a hollow conical section 302 integrally extending from the distal end of the hollow cylindrical section 301 and located outside the outer sheath tube 4, wherein the protective tube 3 has a maximum outer diameter at the proximal end of the hollow conical section 302, so that the protective tube 3 has an arrow shape. The maximum outer diameter of the protective tube 3 is equal to the outer diameter of the outer sheath tube 4, to ensure smooth transition between the protective tube 3 and the outer sheath tube 4 and prevent unsmooth operation of entering/exiting a channel in use owing to an unsmooth surface. The outer circumferential surface of the hollow cylindrical section 301 of the protective tube 3 is close to and tightly fitted with the inner wall of the outer sheath tube 4 for fixation. Preferably, the outer surface of the hollow cylindrical section 301 is provided with protrusions 303 in tight fit with the inner wall of the outer sheath tube 4, or the contact surface between the protective tube 3 and the outer sheath tube 4 is not a smooth surface and can prevent the protective tube 3 from getting loose and separating from the outer sheath tube 4; the protrusions 303 on the protective tube 3 may be barb-shaped protrusions 303 as shown in FIG. 5b, or annular protrusions 303 as shown in FIG. 5c, or rectangular protrusions 303 as shown in FIG. 5d, or protrusions in a shape that is a combination of different shapes. In addition, in the actual operation of injection bulge, the novel medical electrotome is adjusted to a margin electroresection and electrocoagulation state, at this point, the cutting portion at the distal end of the snare ring 1 extends out of the protective tube 3, and cuts out a small incision in the mucosal tissue after being energized, then the snare ring 1 is drawn into the outer sheath tube 4 completely, then the port at the distal end (i.e., the tip end of the arrow head) of the protective tube 3 bears against the incision, so that the injection bulge operation could be performed. Since the port at the distal end of the protective tube 3 is a conical structure with a small diameter, it can be easily inserted into the incision in the mucosal tissue, thereby the injection bulge operation is simple and effective.

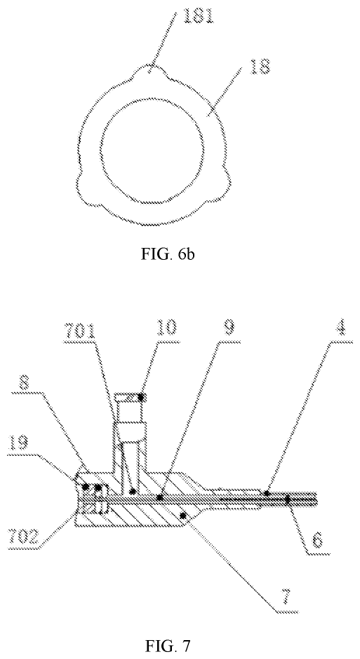

Furthermore, as shown in FIG. 1, the novel medical electrotome further comprises a star-shaped tube 18 arranged inside the outer sheath tube 4 and sleeved on the traction rope 6, and limiting tubes 17 that abut against the two ends of the star-shaped tube 18 and fixed to the traction rope 6, wherein ribs 181 are arranged on the outer circumferential surface of the star-shaped tube 18. The two limiting tubes 17 fix the star-shaped tube 18 and the traction rope 6 in relation to each other to prevent unexpected sliding of the star-shaped tube 18; especially, by the star-shaped tube 18 with ribs 181 being sleeved on the traction rope 6, the traction rope 6 is locally thickened in the radial direction, thereby the clearance between the traction rope 6 and the outer sheath tube 4 is reduced, the stability of axial movement of the traction rope 6 in the outer sheath tube 4 is improved, thus the snare ring 1 can extend out from the distal end of the protective tube 3 stably and thereby the stability of the instrument in the surgical process is ensured, retraction will not easily occur or the delay during extension/retraction is eliminated, and finally the safety of the operation is improved. As shown in FIGS. 6a and 6b, the star-shaped tube 18 is hollow to allow the traction rope 6 to pass through it, and there are 1 to 10 ribs 181 on the outer circumferential surface of the star-shaped tube 18. The maximum outer diameter of the star-shaped tube 18 is smaller than the inner diameter of the outer sheath tube 4, so that the star-shaped tube 18 can move along with the traction rope 6 in the outer sheath tube 4; the non-protruding parts on the outer circumferential surface of the star-shaped tube 18 leave enough channels for the inert gas and ensure the normal use of the electrocoagulation function of the novel medical electrotome. The traction rope 6 and the limiting tubes 17 may be fixed together by compression joint, welding, bonding, or riveting, etc. The star-shaped tube 18 may be made of PP, PTFE, PE, or PA material, so that the star-shaped tube 18 has certain rigidity and lubrication performance, and can be used stably under thin-wall structure.

Furthermore, as shown in FIG. 7, the handle holder 7 has an energized channel 702 that is arranged at the energized connector 11 and receives a portion of the catheter 9, and a sealing device 8 is fixed in the handle holder 7 for isolating the energized channel 702 from the gas-liquid channel 701; the catheter 9 is movably inserted into the sealing device 8, passes through the gas-liquid channel 701 and the central part of the sealing device 8, and then is connected to the energized connector 11, the sealing device 8 prevents the inert gas or liquid in the gas-liquid channel 701 from leaking to the energized channel 702, and ensures that the inert gas or liquid in the gas-liquid channel 701 flows along the clearance between the outer sheath tube 4 and the traction rope 6 toward the distal end. Preferably, the sealing device 8 is composed of 1 to 8 O-rings arranged side by side in the extension direction of the catheter 9, the plurality of O-rings are mounted side by side in the inner hole of the handle holder 7 and fixed by a non-metal pressing block 19, the pressing block 19 may be fixed in the handle holder 7 by bonding, ultrasonic welding, riveting, or compression joint, and applies certain axial pressure to the plurality of O-rings, so that the O-rings deform after being pressed and seal the catheter 9 inserted through their inner holes.

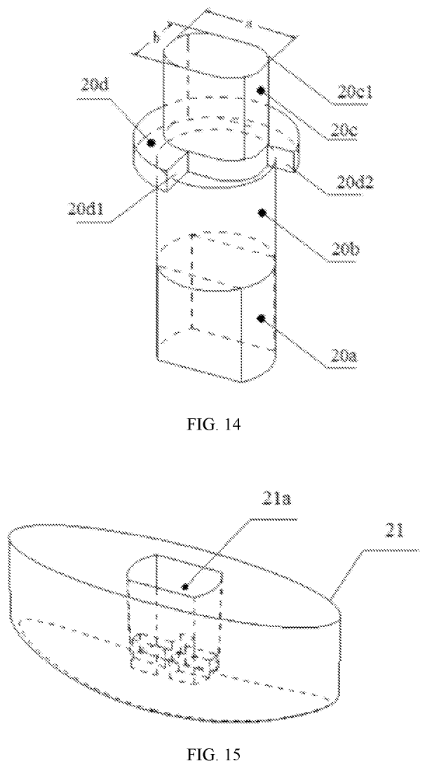

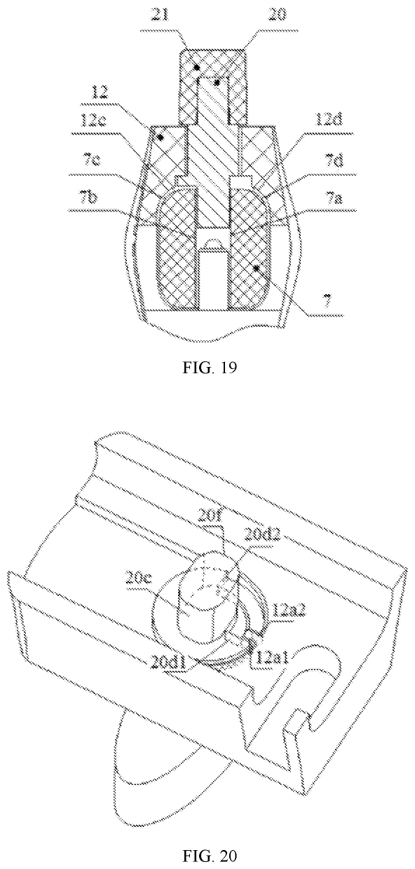

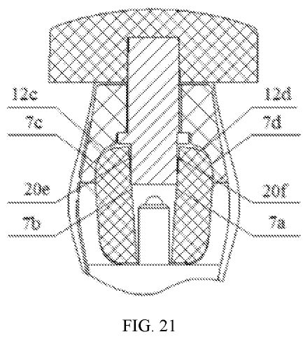

Furthermore, the handle assembly in the present application is a lockable handle assembly, which can ensure the extension length of the cutting knife 16 and the snare ring 1 and improve the operation and service performance of the novel medical electrotome. Specifically, as shown in FIGS. 13-21, the lockable handle assembly further comprises a locking assembly, in addition to the operating handle 12 and the handle holder 7; wherein the operating handle 12 has a through-hole 12b perpendicular to the sliding direction of the operating handle 12; the handle holder 7 has an open slot extending axially in the sliding direction of the operating handle 12; the locking assembly comprises a rotating rod 20 inserted in the through-hole 12b, with an end 20c of the rotating rod 20 located in the open slot, as shown in FIG. 20; in addition, the end 20c of the rotating rod 20 has two opposite long planar sides, and the length a of the long sides is greater than the length b of the other sides. Particularly, when the other sides of the rotating rod 20 are oriented in the width direction of the opening of the open slot, as shown in FIG. 19, the operating handle 12 can move axially in relation to the handle holder 7; when the rotating rod 20 is rotated so that the long sides of the rotating rod 20 are oriented in the width direction of the opening of the open slot, as shown in FIG. 21, the rotating rod 20 will enlarge the opening degree of the open slot, thus the outer wall surfaces 7c and 7d of the handle holder 7 tightly abut against the wall faces 12c and 12d of the operating handle 12 with through-hole, thereby the handle holder 7 and the operating handle 12 are tightly locked.

Therefore, when there is a need to push or pull, the other sides of the rotating rod 20 are oriented in the width direction of the opening of the open slot of the handle holder 7 by means of the locking assembly, thus the handle holder 7 can move axially in relation to the operating handle 12 and push or pull is realized. When locking positioning is required, the rotating rod 20 can be rotated so that the long sides of the rotating rod 20 are oriented in the width direction of the opening of the open slot, the rotating rod 20 presses the handle holder 7 so that the open slot of the handle holder 7 is enlarged, thereby the handle holder 7 is tightly fitted with the operating handle 12, and the handle holder 7 and the operating handle 12 are locked together and can't move axially in relation to each other, i.e., the relative position of the handle holder 7 is locked.

Preferably, as shown in FIGS. 13 and 20, the locking assembly further comprises a rotating block 21, the rotating block 21 is fixedly connected with the rotating rod 20, for example, by compression joint, riveting, ultrasonic welding, bonding, or snap-fitting. In this example, the rotating rod 20 is divided into three sections, i.e., a cylindrical middle section 20b, a terminal section 20a arranged on top of the middle section 20b, and a terminal section 20c arranged at the bottom of the middle section 20b, wherein both the terminal section 20a and the terminal section 20c are both flat; the terminal section 20a at the top of the rotating rod 20 constitutes a positioning section of the rotating rod 20, and the terminal section 20a is stuck in a clamping hole 21a in the rotating block 21, so that the rotating rod 20 can be driven to rotate by turning the rotating block 21 to facilitate operation; the terminal section 20c at the bottom of the rotating rod 20 constitutes a locking section of the rotating rod 20, and is inserted in the above-mentioned open slot of the handle holder 7.

Specifically, as shown in FIGS. 18 and 20, in this example, an end of the rotating rod 20 is further provided with two opposite planar short sides 20e and 20f, and the short sides 20e and 20f are connected with the long sides via arc surfaces 20c1; when the short sides 20e and 20f are oriented in the width direction of the opening of the open slot, the handle holder 7 can move axially in relation to the operating handle 12. In this example, with the arrangement of the short sides, arc surfaces, and long sides, the rotating rod 20 can be locked and positioned conveniently, and can also be rotated conveniently.

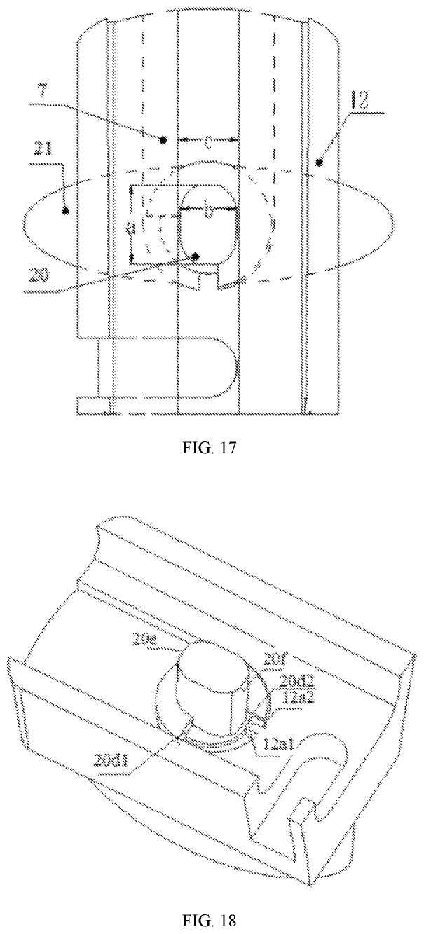

As shown in FIG. 17, with regard to the length c of the open slot of the handle holder 7, the length b of the short sides 20e and 20f of the rotating rod 20, and the length a of the long sides of the rotating rod 20, it may also be understood that the spacing between the two short sides 20e and 20f is a and the spacing between the two long sides is b, and a>c>b; transition arcs (i.e., the arc surfaces 20c1 described above) arranged between the long sides and the short sides can facilitate smooth rotation of the rotating rod 20 and enlarge the open slot of the handle holder 7 outward.

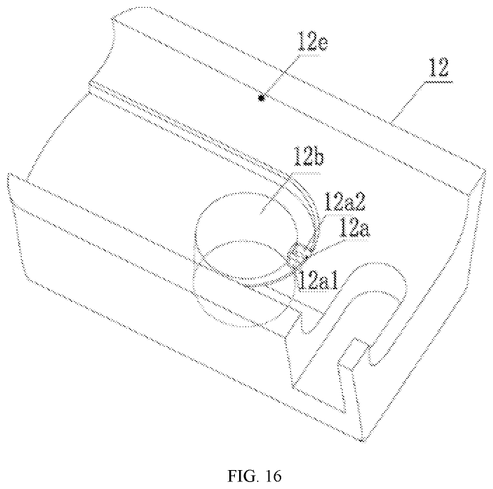

For better positioning, as shown in FIGS. 16 and 20, the outer circumference wall of the rotating rod 20 has a round of circumferential flange 20d with an opening, the operating handle 12 has a raised limiting part 12a arranged at the through-hole 12b and located in the opening of the circumferential flange 20d; in the rotation process of the rotating rod 20, when an end face 20d2 of the circumferential flange 20d bears against a side 12a2 of the limiting part 12a, as shown in FIGS. 18 and 19, the other sides of the rotating rod 20 are oriented in the width direction of the opening of the open slot; as shown in FIGS. 20 and 21, when the other end face 20d1 of the circumferential flange 20d bears against the other side 12a1 of the limiting part 12a, the long sides of the rotating rod 20 are oriented in the width direction of the opening of the open slot. In this example, the circumferential flange 20d of the rotating rod 20 is near the above-mentioned locking section to realize stopping and limiting, so as to prevent the rotating rod 20 from falling from the through-hole 12b and avoiding a poor locking effect incurred by an excessive large rotation angle of the rotating rod 20.

In use, the handle assembly in this example turns the rotating block 21 to drive the rotating rod 20 to rotate synchronously, as shown in FIGS. 20 and 21, till the end face 20d1 of the rotating rod 20 bears against the side 12a1 of the operating handle 12 and stops rotation; at that point, the short sides 20e and 20f of the rotating rod 20 support the surfaces 7b and 7a of the handle holder 7 respectively, so that the handle holder 7 is pushed open laterally, and then the surfaces 7c and 7d of the handle holder 7 support the surfaces 12c and 12d of the operating handle 12 respectively, thereby an effect that the handle holder 7 is locked by the rotating rod 20 and the operating handle 12 is attained; when the rotating block 21 is rotated in the reverse direction to drive the rotating rod 20 to rotate in the reversed direction, as shown in FIGS. 18 and 19, till the end face 20d2 bears against the side 12a2 of the operating handle 12 and stops rotation, so that the handle holder 7 is unlocked. Thus, the handle holder 7 can be locked, and the requirement for the user to push/pull the handle holder 7 by a certain length is met, so that the electric knife can be locked in position when the electric knife extends to a certain length.

In summary, when the novel medical electrotome is used, the doctor may push the operating handle 12 to slide back and forth on the handle holder 7 and control the positions of the snare ring 1 and the cutting knife 16 as required, and then can connect the high frequency power source 13, connect the gas storage tank 14, and connect the syringe 15 as required for injection operation, electroresection operation, electrocoagulation operation, and margin electroresection and electrocoagulation operation, thereby execute functions including marking of resection range, injection bulge, mucosal incision and resection, and hemostasis, and finally accomplish an ESD surgical operation completely, independently and safely, without exchange of instruments such as syringe needles for injection bulge, etc. Therefore, the novel medical electrotome has integrated a variety of functions and has a simple structure, can be operated safely and conveniently, and can shorten the operation time, reduces the difficulties of the operation, and save the treatment cost. Therefore, the present invention effectively overcome the disadvantages in the prior art, and has a high value of industrial utilization.

The above examples only illustrate the principle and efficacy of the present invention, but are not intended to limit the present invention. Those skilled in the art can make various modifications or changes to the above examples without departing from the spirit and scope of the present invention. Therefore, all equivalent modifications or changes made by those having ordinary knowledge in the art without departing from the spirit and technical idea disclosed in the present invention shall still be deemed as falling in the scope of the claims of the present invention.

1. A knife head assembly of a novel medical electrotome, comprising a snare ring and a cutting knife that comprises a knife handle and a knife head, wherein, the surface of the knife handle is provided with two grooves arranged symmetrically and extending in a length direction of the knife handle, a distal end of the groove is provided with a through-hole penetrating through the knife handle, and two ends of the through-hole are in communication with the two grooves respectively; a distal end of the snare ring is integrally provided with a ring connection portion formed by bending, the ring connection portion comprises a first bent section, a second bent section and a third bent section that are connected sequentially, wherein the second bent section is inserted through the through-hole, and the first bent section and the third bent section are limited in the two grooves respectively, so that the distal end of the snare ring is embedded and fixed in the knife handle.

2. The knife head assembly according to claim 1, wherein the snare ring is formed by multiple strands of braided wires made of stainless steel, or multiple strands of braided wires made of nickel and titanium, or multiple strands of braided wires made of a composite material of stainless steel, nickel and titanium, or a single wire made of stainless steel, or a single wire made of nickel and titanium.

3. The knife head assembly according to claim 1, wherein the snare ring and the knife handle are fixed together by welding or bonding.

4. The knife head assembly according to claim 1, wherein the cutting knife is an integrally formed part.

5. The knife head assembly according to claim 1, wherein the knife head is of a mushroom-head type, a doornail type, a triangular type, an L-type or a linear type.

6. A novel medical electrotome, comprising an outer sheath tube, a traction rope movably inserted in the outer sheath tube, and a handle assembly configured to drive the traction rope to move, wherein, the novel medical electrotome further comprises the knife head assembly according to claim 1, and a proximal end of the snare ring is fixedly connected with a distal end of the traction rope.

7. The novel medical electrotome according to claim 6, further comprising an injection unit, an electroresection unit, an electrocoagulation unit, and a margin electroresection unit.

8. The novel medical electrotome according to claim 7, wherein the injection unit comprises an outer sheath tube, a protective tube fixed to a distal end of the outer sheath tube, a handle holder fixed with a proximal end of the outer sheath tube, a catheter movably inserted in the handle holder and fixed with the proximal end of the traction rope, a gas-liquid connector arranged on the handle holder, and a gas-liquid channel arranged in the gas-liquid connector.

9. The novel medical electrotome according to claim 8, wherein the electroresection unit comprises a connecting tube arranged inside the outer sheath tube, an operating handle in sliding fit with the handle holder in the extension direction of the outer sheath tube, an energized connector arranged on the operating handle and connected with the catheter, the catheter, the handle holder, the outer sheath tube, the traction rope, the snare ring and the cutting knife.

10. The novel medical electrotome according to claim 9, wherein the electrocoagulation unit comprises electrocoagulation electrodes fixed in the outer sheath tube, the energized connector, the operating handle, the catheter, the gas-liquid connector, the handle holder, the gas-liquid channel, the outer sheath tube, the traction rope, the connecting tube, the snare ring, the cutting knife and the protective tube.

11. The novel medical electrotome according to claim 10, wherein the margin electroresection unit comprises the energized connector, the operating handle, the catheter, the handle holder, the outer sheath tube, the traction rope, the connecting tube, the snare ring and the cutting knife.
